(19) United States
(12) Patent Application Publication
(10) Pub. No.: US 2011/0263902 A1
(43) Pub. Date: Oct. 27, 2011

(54) PROCESS FOR PREPARING 4,4'-DICHLORODIPHENYL SULFONE

(75) Inventors: Patrick Deck, Sao Paulo City (BR); Heiner Schelling, Kirchheim (DE); Florian Garlichs, Neustadt (DE)

(73) Assignee: BASF SE, Ludwigshafen (DE)

(21) Appl. No.: 13/090,711

(22) Filed: Apr. 20, 2011

Related U.S. Application Data
(60) Provisional application No. 61/326,688, filed on Apr. 22, 2010.

(51) Int. Cl. C07C 315/00

(52) U.S. Cl. 568/34

(57) ABSTRACT

The invention relates to a process for preparing 4,4′-dichlorodiphenyl sulfone, comprising the reaction of monochlorobenzene and liquid sulfur trioxide, wherein the liquid sulfur trioxide used has a boron content of at most 100 ppm based on the total weight of the sulfur trioxide used, including all secondary components.

The invention relates to a process for preparing 4,4′-dichlorodiphenyl sulfone, comprising the reaction of monochlorobenzene and liquid sulfur trioxide, wherein the liquid sulfur trioxide used has a boron content of at most 100 ppm based on the total weight of the sulfur trioxide used, including all secondary components.

4,4′-Dichlorodiphenyl sulfone is used especially as a monomer in the synthesis of polyarylene ether sulfones. Examples of commercial significance are polyether sulfone (polymerization of 4,4′-dihydroxydiphenyl sulfone with 4,4′-dichlorodiphenyl sulfone), polysulfone (polymerization of bisphenol A with 4,4′-dichlorodiphenyl sulfone) and polyphenylene sulfone (polymerization of 4,4′-dihydroxybiphenyl with 4,4′-dichlorodiphenyl sulfone). 4,4′-Dichlorodiphenyl sulfone is thus a central element for the preparation of these industrial polymers.

The preferred reactant for the preparation of polyarylene ether sulfones is high-purity 4,4′-dichlorodiphenyl sulfone, firstly since the 4,4′ isomer forms exclusively linear, nonangular polymers which have the desired product properties, for example chemical and thermal stability, high dimensional stability and low flammability, and secondly since impurities frequently lead to undesired discoloration and to a deterioration in the properties of the polymers.

Processes for preparing 4,4′-dichlorodiphenyl sulfone proceeding from monochlorobenzene are known from the prior art. The known processes comprise, more particularly, the preparation proceeding from monochlorobenzene and a sulfonating agent via 4-chlorobenzenesulfonic acid as an intermediate which is generally not isolated.

U.S. Pat. No. 2,593,001 describes a continuous process for preparing diaryl sulfones by reaction of aromatic sulfonic acids with aromatics, wherein the water of reaction is removed continuously from the reaction zone by the aromatic compound added in gaseous form in countercurrent. U.S. Pat. No. 4,937,387 likewise discloses the sulfonation of monochlorobenzene by means of sulfur trioxide to form chlorobenzenesulfonic acid.

U.S. Pat. No. 2,971,985 discloses the synthesis of dichlorodiphenyl sulfone using SO₃, dimethyl sulfate and monochlorobenzene. EP 0 381 049 A1 likewise describes a process for preparing 4,4′-dichlorodiphenyl sulfone. This involves reacting sulfur trioxide, dimethyl sulfate and chlorobenzene at 50 to 100° C.

The use of SO₃ offers clear advantages over other sulfonating agents such as sulfuric acid or oleum by virtue of the higher reactivity.

As is known, sulfur trioxide exists in three polymorphs. Cooling of gaseous SO₃ forms the γ-SO₃ polymorph, which melts at 16.9° C. and boils at 44.5° C. When γ-SO₃ is stored below 29° C. for a prolonged period, it solidifies and is converted to the polymeric asbestos-like α-SO₃ and β-SO₃ polymorphs (melting point: α-SO₃: 62.2° C. β-SO₃: 30.5° C.). When handling on the industrial scale, this polymerization should be absolutely prevented, since the solidified SO₃ can be melted again only with difficulty, if at all, in apparatus, transport containers and pipelines. Since the melting of the polymeric polymorphs can result in an abrupt, explosive rise in pressure, the prevention of polymerization is highly relevant to safety.

SO₃ supplied commercially and/or stored at temperatures below 30° C. is always protected with a stabilizer against polymerization for this reason. Numerous stabilizers are known. Typically, stabilizers which are effective even in small amounts, can be metered into the SO₃ in a simple manner and do not require any further process step (for example heating) to be effective are used. For this purpose, preference is given to using organic sulfur compounds such as dimethyl sulfate (GB 735 836) or dimethyl sulfoxide (U.S. Pat. No. 2,820,697), and boron compounds such as nitrosyl tetrafluoroborate (U.S. Pat. No. 2,805,126), methyl borate or boron trifluoride dimethyl etherate (both U.S. Pat. No. 2,492,706). The abovementioned U.S. Pat. No. 2,971,985 describes the use of stabilized sulfur trioxide.

Boron trifluoride dimethyl etherate is particularly frequently used as a stabilizer, since even small amounts (for example 0.1 to 1% by weight based on the total weight of the sulfur trioxide used, including all secondary components) very effectively suppress conversion to the α-SO₃ and β-SO₃ polymorphs.

The syntheses for preparation of dichlorodiphenyl sulfone not only form the desired 4,4′-dichlorodiphenyl sulfone, but different amounts of the 2,4′ and of the 3,4′ isomer are always obtained, which are referred to collectively hereinafter as incorrect isomers of the 4,4′-dichlorodiphenyl sulfone. In order to arrive at a 4,4′-dichlorodiphenyl sulfone usable in polymerizations, it has to be isolated in very pure form (typically>99% by weight).

The use of liquid SO₃ stabilized with boron compounds leads, however, in the sulfonation of monochlorobenzene with the sulfur trioxide mentioned, to significantly poorer yields of the desired 4,4′-dichlorodiphenyl sulfone isomer relative to the aforementioned incorrect isomers. In the case of isolation of chlorobenzenesulfonic acid as an intermediate, when liquid SO₃ stabilized with boron compounds is used, a significantly poorer yield of the desired 4-chlorobenzenesulfonic acid isomer relative to 2- or 3-chlorobenzenesulfonic acid is obtained, which in turn lead to the aforementioned incorrect isomers of the dichlorodiphenyl sulfone.

In order to obtain 4,4′-dichlorodiphenyl sulfone in a quality needed for use as a monomer unit, a workup of the crude product initially obtained, i.e. a mixture comprising 4,4′-dichlorodiphenyl sulfone, is thus always required. For this purpose, the prior art discloses different processes.

U.S. Pat. No. 4,937,387 describes, building on the synthesis according to U.S. Pat. No. 2,593,001, the separation of the reaction mixture by addition of water, separation of the two liquid phases formed and subsequent isolation of dichlorodiphenyl sulfone. U.S. Pat. No. 4,016,210 describes the crystallization of 4,4′-dichlorodiphenyl sulfone from a reaction mixture which results from the reaction of chlorobenzenesulfonic acid and chlorobenzene.

However, the complexity of the workup is determined to a high degree by the amount of incorrect isomers of 4,4′-DCDPS. Minimization of the content of the incorrect isomers is thus desirable.

Mixtures of the dichlorodiphenyl sulfone isomers can be worked up, for example, by crystallization with/from alcohols, such that increased purities of the desired 4,4′-dichlorodiphenyl sulfone are obtained. EP-A 279 387 describes this type of purification by recrystallization.

A further means of removing the incorrect isomers is the chromatographic separation of the isomer mixture described in U.S. Pat. No. 4,876,390.

For a high quality of the polymer, a low color number and in particular the isomeric purity of the 4,4′-dichlorodiphenyl sulfone used are essential.

It was thus an object of the present invention to discover a process for preparing 4,4′-dichlorodiphenyl sulfone, which provides 4,4′-dichlorodiphenyl sulfone in high purity in a simple manner in process technology terms. The proportion of incorrect isomers of 4,4′-dichlorodiphenyl sulfone formed in the preparation should be reduced compared to the prior art. Any workup required to obtain 4,4′-dichlorodiphenyl sulfone in pure form should be performable with standard processes in a very simple manner.

The aforementioned objects are achieved by the process according to the invention for preparing 4,4′-dichlorodiphenyl sulfone. Preferred embodiments can be inferred from the claims and the description which follows. Combinations of preferred embodiments do not leave the scope of the present invention.

According to the invention, the present process for preparing 4,4′-dichlorodiphenyl sulfone comprises the reaction of monochlorobenzene and liquid sulfur trioxide, wherein the liquid sulfur trioxide used has a boron content of at most 100 ppm based on the total weight of the liquid sulfur trioxide used, including all secondary components.

In the context of the present invention, the boron content is always calculated as the weight of the boron atoms (in the ppm unit, which denotes parts by weight in the context of the present invention) and is based on the total weight of the liquid sulfur trioxide used, including all secondary components. In the context of the present invention, the boron content is determined quantitatively by means of mass spectrometry with inductively coupled plasma (ICP-MS). The detection limit typically achievable is below 1 ppb.

If present, boron in the context of the present invention is in the form of a boron compound or in the form of boron compounds in the liquid sulfur trioxide used. In the context of the present invention, a boron compound is understood to mean an inorganic or organic compound which comprises at least one boron atom, especially an organic boron compound.

Boron compounds are customary as stabilizers of sulfur trioxide. Stabilized sulfur trioxide is generally understood to mean that which is stabilized by suitable stabilizers. Stabilizers are therefore additives which, in suitable amounts, delay or prevent the conversion of the γ-polymorph to the α- or β-polymorph. Unstabilized sulfur trioxide is that which does not comprise any stabilizers.

The provision of liquid sulfur trioxide used in accordance with the invention is explained below in the context of step (a) of a preferred embodiment.

The liquid sulfur trioxide used preferably has a boron content of at most 50 ppm based on the total weight of the sulfur trioxide used, including all secondary components.

The liquid sulfur trioxide used more preferably has a boron content of at most 20 ppm based on the total weight of the sulfur trioxide used, including all secondary components.

The liquid sulfur trioxide used most preferably has a boron content of 1 ppb to 10 ppm based on the total weight of the sulfur trioxide used, including all secondary components. A lower limit with regard to the content of boron compounds arises in the context of the present invention merely in that complete freedom from boron compounds is achievable only with great complexity, if at all. The lower limit is thus, for example, 1 ppb or 0.01 ppm, especially 0.1 ppm.

Boron compounds which can be used as stabilizers are especially nitrosyl tetrafluoroborate, boron oxide, boron trichloride, boron trifluoride, borax (NaB₄O₇), sodium tetrafluoroborate, potassium tetrafluoroborate, iron(II) tetrafluoroborate, orthoboric acid, metaboric acid, alkyl borates and boron trifluoride dialkyl etherates. It is preferred when the boron content, owing to the aforementioned boron compounds, is at most 80 ppm in total, more preferably at most 50 ppm, especially at most 20 ppm, based on the total weight of the sulfur trioxide used, including all secondary components.

In the context of the present invention, the liquid sulfur trioxide is preferably used in the γ-polymorph. Liquid sulfur trioxide in the γ-polymorph consists of monomeric SO₃ and of cyclic trimeric SO₃. It is thus characterized by absence of linear oligomeric SO₃.

In addition, it is preferred when the sulfur trioxide used has a purity of at least 99.7% by weight, more preferably at least 99.8% by weight, especially 99.9% by weight, most preferably at least 99.99% by weight, based in each case on the total weight of the sulfur trioxide used in the reaction.

Purity is understood to mean the content of sulfur trioxide relative to the total amount of the sulfur trioxide used in the reaction, including all secondary components. Secondary components are all other compounds in the sulfur trioxide used except sulfur trioxide.

In a preferred embodiment, the process according to the invention comprises the following steps:
(a) providing liquid sulfur trioxide as defined above, and then
(b) converting the liquid sulfur trioxide obtained in step (a) and monochlorobenzene to a mixture comprising 4,4′-dichlorobenzenesulfone.

The liquid sulfur trioxide is preferably exclusively at a temperature of at least 30° C. between steps (a) and (b).

In a preferred embodiment, step (b) is followed, in a subsequent step (c), by the removal of 4,4′-dichlorodiphenyl sulfone from the mixture obtained in step (b).

The individual steps are explained hereinafter.

### Step (a)

Methods for purifying the sulfur trioxide with removal of boron compounds are known per se to those skilled in the art. It is essential to the invention that the sulfur trioxide used in step (b) has the abovementioned inventive or preferred properties.

It is especially possible to stabilize liquid sulfur trioxide by means of compounds other than boron compounds, and to use it in the process according to the invention. Suitable stabilizers are, for example, organic compounds, for example ethylene, dimethyl ether, diethyl ether, ethyl methyl ether, carboxylic esters, formamide, and especially organic sulfur compounds which have low or zero Lewis acidity, especially dimethyl sulfate and dimethyl sulfoxide, sulfonic esters or sulfonamides. In the context of the present invention, particular preference is given to the conversion of dimethyl sulfate-stabilized liquid sulfur trioxide.

If unstabilized sulfur trioxide is used, preference is given to keeping it exclusively at a temperature of at least 30° C. between the preparation thereof and use thereof.

The liquid sulfur trioxide used in accordance with the invention with a boron content of at most 100 ppm based on the total weight of the sulfur trioxide used, including all secondary components, can alternatively be taken from a heated pipeline or a heated reservoir or transport vessel, especially in unstabilized form.

When the sulfur trioxide for use does not satisfy the properties required in accordance with the invention, removal of the boron compounds is required. The liquid sulfur trioxide can be purified by different processes known to those skilled in the art. Preference is given to purifying the sulfur trioxide by distillation to remove boron compounds in step (a). Corresponding distillative processes are known to those skilled in the art. Sulfur trioxide can be distilled especially out of oleum or out of stabilized sulfur trioxide.

Options for distillative purification of liquid sulfur trioxide for the purpose of removing boron compounds are especially a batch distillation or a continuous distillation. In the batch distillation, the distillation time is matched to the reaction of monochlorobenzene and liquid sulfur trioxide, which is in that case preferably likewise conducted in a batch process. The batch distillation is preferably conducted in such a way that it has ended shortly before completion of the parallel reaction of monochlorobenzene and liquid sulfur trioxide, and a sufficient amount of SO₃ for the next synthesis batch is available with the completion of the distillation. In the continuous distillation, the throughput is determined by the time required for the reaction of monochlorobenzene and liquid sulfur trioxide. It is adjusted such that, on completion of one batch for reaction of monochlorobenzene and liquid sulfur trioxide, a sufficient amount of SO₃ for the next batch is available and hence no long dead times or storage times arise between production and consumption. When the dichlorodiphenyl sulfone synthesis is conducted continuously, continuous distillation is preferred. The liquid sulfur trioxide purified by distillation can be used especially in the process according to U.S. Pat. No. 2,593,001.

### Step (b)

Processes for preparing 4,4′-dichlorodiphenyl sulfone proceeding from monochlorobenzene and sulfur trioxide are known per se and can be implemented as process steps (b) of the present process.

The step (b) mentioned relates to the conversion of the liquid sulfur trioxide obtained in step (a) and monochlorobenzene to a mixture comprising 4,4′-dichlorodiphenyl sulfone (crude product). The by-products formed in the conversion proceeding from monochlorobenzene are especially 2,4′-dichlorodiphenyl sulfone and/or 3,4′-dichlorodiphenyl sulfone (incorrect isomers of 4,4′-dichlorodiphenyl sulfone). In addition, 2-chlorobenzenesulfonic acid, 3-chlorobenzenesulfonic acid and/or 4-chlorobenzenesulfonic acid are generally formed.

In principle, in the context of the process according to the invention, useful processes are all known processes for preparing 4,4′-dichlorodiphenyl sulfone which proceed from monochlorobenzene and use sulfur trioxide as the sulfonating agent. Corresponding processes are known per se to those skilled in the art.

In a preferred embodiment, monochlorobenzene is first reacted with the liquid sulfur trioxide to form 4-chlorobenzenesulfonic acid. The liquid sulfur trioxide is mixed with the monochlorobenzene by customary processes known to those skilled in the art. 4-Chlorobenzenesulfonic acid is formed with no further assistance. The person skilled in the art will aim to provide measures for removing the heat of reaction which arises.

Subsequently, 4,4′-dichlorodiphenyl sulfone is prepared proceeding from 4-chlorobenzenesulfonic acid in the manner known to those skilled in the art, for example by reaction of the 4-chlorobenzenesulfonic acid with monochlorobenzene in a countercurrent column, in which case the water of reaction is stripped out continuously overhead by the aromatic added in gaseous form in the bottom of the column. For the synthesis of 4,4′-dichlorodiphenyl sulfone, 4-chlorobenzenesulfonic acid or else sulfuric acid can be added at the top of the column. The latter reacts in the column with monochlorobenzene first to give monochlorobenzenesulfonic acid, which subsequently likewise reacts with monochlorobenzene to give dichlorodiphenyl sulfone. The corresponding process is described, for example, in U.S. Pat. No. 2,593,001, the content of which is hereby fully incorporated. In addition, the process according to U.S. Pat. No. 4,937,387 can be used, the content of which is hereby likewise fully incorporated.

In an alternative embodiment, dichlorodiphenyl sulfone is prepared using SO₃, dimethyl sulfate and monochlorobenzene. Preferably, SO₃ and dimethyl sulfate are first allowed to react under moderate conditions in a molar ratio of 2 to 1.

In the course of this, some of the SO₃ reacts with dimethyl sulfate to form the corresponding pyrosulfate. The rest of the SO₃ remains dissolved in the liquid which forms. This mixture is subsequently added at temperatures below 100° C. to 2 mol of monochlorobenzene per 2 mol of SO₃ and 1 mol of dimethyl sulfate. The dissolved SO₃, the dimethylpyrosulfate and the monochlorobenzene form 1 mol of dichlorodiphenyl sulfone and 2 mol of monomethyl sulfate. The reaction mixture is subsequently passed into water. Dichlorodiphenyl sulfone precipitates out. It is filtered off and dried. The corresponding process is described, for example, in U.S. Pat. No. 2,971,985, the content of which is hereby fully incorporated. However, the person skilled in the art is not restricted to the aforementioned methods with regard to the workup method.

### Step (c)

In the course of step (c), 4,4′-dichlorodiphenyl sulfone is preferably removed from the mixture obtained in step (b), i.e. the crude product which comprises the desired reaction product and by-products is worked up.

Processes for removing 4,4′-dichlorodiphenyl sulfone from the mixture obtained in step (b) are known per se to those skilled in the art.

In one embodiment, the reaction mixture is separated by adding water and separating the two liquid phases which form. The aqueous phase comprises unconverted monochlorobenzenesulfonic acid. The water is evaporated off and the monochlorobenzenesulfonic acid is recovered as a feedstock. Dichlorodiphenyl sulfone can be isolated from the organic phase, which consists predominantly of monochlorobenzene and dichlorodiphenyl sulfone. A corresponding process is described, for example, in U.S. Pat. No. 4,937,387, the content of which is hereby fully incorporated.

4,4′-Dichlorodiphenyl sulfone can be removed from the crude product, for example, by chromatography. The removal is preferably effected by recrystallization, as described, for example, in EP 279 387, the content of which is hereby fully incorporated.

The present invention further provides for the use of liquid sulfur trioxide with the properties required or preferred in accordance with the invention for preparing 4,4′-dichlorodiphenyl sulfone.

### EXAMPLES

The selectivities of formation of the isomers were determined by high-performance liquid chromatography (HPLC) with pure substances (2-chlorobenzenesulfonic acid, 4-chlorobenzenesulfonic acid, 2,4′-dichlorodiphenyl sulfone, 4,4′-dichlorodiphenyl sulfone) for calibration. A column of the Purospher RP-18 endcapped, 5 μm, 250*3 mm type was used. The eluents used were the following compounds: A) water/phosphoric acid (85%) 1000/1 (v/v), B) acetonitrile/phosphoric acid (85%) 1000/1 (v/v). The gradient was selected as follows:

| Min | 0 | 15 | 40 | 41 |
|---|---|---|---|---|
| % A) | 95 | 45 | 45 | 95 |
| % B) | 5 | 55 | 55 | 5 |

The flow rate was 0.7 ml/min; detection: 226 nm; injection volume: 10 μl.

### Example 1

### Comparative Example

Use of sulfur trioxide stabilized with BF₃.OMe₂ (0.3% by weight of BF₃.OMe₂ based on the total weight of sulfur trioxide) in the synthesis of dichlorodiphenyl sulfone according to U.S. Pat. No. 2,971,985

126.1 g (1 mol) of dimethyl sulfate were heated to 70-75° C. with exclusion of air humidity and 80.1 g (1 mol) of liquid sulfur trioxide stabilized with 0.3% by weight of boron trifluoride dimethyl etherate were added at this temperature. The mixture was left to stir at this temperature for 30 min and then cooled to 20° C. A further 80.1 g (1 mol) of liquid sulfur trioxide stabilized with 0.3% by weight of boron trifluoride dimethyl etherate were added at such a rate that the temperature of 30° C. was not exceeded. The reaction mixture was added within 20 min to 225.1 g (2 mol) of chlorobenzene preheated to 50° C. Subsequently, the mixture was stirred at 50° C. for another 1 h. An HPLC chromatogram showed an isomer ratio of the 4,4′-dichlorodiphenyl sulfone target product to the 2,4′-dichlorodiphenyl sulfone by-product of 15:1.

### Example 2

Use of sulfur trioxide stabilized with dimethyl sulfate (1% by weight based on the total weight of sulfur trioxide) in the synthesis of dichlorodiphenyl sulfone according to U.S. Pat. No. 2,971,985

126.1 g (1 mol) of dimethyl sulfate were heated to 70-75° C. with exclusion of air humidity and 80.1 g (1 mol) of liquid sulfur trioxide stabilized with 1% by weight of dimethyl sulfate (boron-free) were added at this temperature. The mixture was left to stir at this temperature for 30 min and then cooled to 20° C. A further 80.1 g (1 mol) of liquid sulfur trioxide stabilized with 1% by weight of dimethyl sulfate were added at such a rate that the temperature of 30° C. was not exceeded. The reaction mixture was added within 20 min to 225.1 g (2 mol) of chlorobenzene preheated to 50° C. Subsequently, the mixture was stirred at 50° C. for another 1 h. An HPLC chromatogram showed an isomer ratio of the 4,4′-dichlorodiphenyl sulfone target product to the 2,4′-dichlorodiphenyl sulfone by-product of 32:1.

### Example 3

Use of unstabilized sulfur trioxide and sulfur trioxide stabilized with BF₃-dimethyl etherate in the sulfonation of chlorobenzene as a precursor of the preparation of 4,4′-dichlorodiphenyl sulfone

64 g (0.8 mol) of sulfur trioxide which has been stabilized with different amounts of boron trifluoride dimethyl etherate was added with exclusion of air humidity at a maximum of 40° C. to 400 g (3.55 mol) of chlorobenzene within 120 min. The resulting solution can be used for synthesis of dichlorodiphenyl sulfone, for example according to U.S. Pat. No. 2,593,001. The ratio of the chlorobenzenesulfonic acid isomers formed as a function of the stabilizer content can be found in the table which follows. Boron-free sulfur trioxide was obtained in example 3 by distillation out of oleum.

TABLE 1

| | Boron content [ppm] from BF₃-DME | | | | |
|---|---|---|---|---|---|
| | 0 | 50 | 100 | 300 | 500 |
| Ratio of 4-chloro- | 95:1 | 92:1 | 89:1 | 60:1 | 61:1 |
| benzenesulfonic acid to | | | | | |
| 2-chlorobenzenesulfonic | | | | | |
| acid | | | | | |

1.-10. (canceled)

11. A process for preparing 4,4′-dichlorodiphenyl sulfone, comprising reacting monochlorobenzene and liquid sulfur trioxide, wherein the liquid sulfur trioxide used has a boron content of at most 100 ppm based on the total weight of the sulfur trioxide used, including all secondary components.

12. The process according to claim 11, wherein the liquid sulfur trioxide used has a boron content of at most 50 ppm based on the total weight of the sulfur trioxide used, including all secondary components.

13. The process according to claim 11, wherein the liquid sulfur trioxide used has a boron content of at most 20 ppm based on the total weight of the sulfur trioxide used, including all secondary components.

14. The process according to claim 11, wherein the liquid sulfur trioxide used has a boron content of 1 ppb to 10 ppm based on the total weight of the sulfur trioxide used, including all secondary components.

15. The process according to claim 11, wherein the liquid sulfur trioxide used is in the γ-polymorph and has a purity of at least 99.7% by weight, based on the total weight of the sulfur trioxide used, including all secondary components.

16. The process according to claim 11, wherein the liquid sulfur trioxide used is in the γ-polymorph and has a purity of at least 99.9% by weight, based on the total weight of the sulfur trioxide used, including all secondary components.

17. The process according to claim 14, wherein the liquid sulfur trioxide used is in the γ-polymorph and has a purity of at least 99.9% by weight, based on the total weight of the sulfur trioxide used, including all secondary components.

18. A process for preparing 4,4′-dichlorodiphenyl sulfone comprising
(a) providing liquid sulfur trioxide wherein the liquid sulfur trioxide used has a boron content of at most 100 ppm based on the total weight of the sulfur trioxide used, including all secondary components, and then
(b) converting the liquid sulfur trioxide obtained in step (a) and monochlorobenzene to a mixture comprising 4,4′-dichlorodiphenyl sulfone.

19. The process according to claim 18, wherein the liquid sulfur trioxide is kept constantly at a temperature of at least 30° C. between steps (a) and (b).

20. The process according to claim 18, wherein step (b) is followed, in step (c), by the removal of 4,4′-dichlorodiphenyl sulfone from the mixture obtained in step (b).

21. The process according to claim 19, wherein step (b) is followed, in step (c), by the removal of 4,4′-dichlorodiphenyl sulfone from the mixture obtained in step (b).

22. The process according to claim 18, wherein the liquid sulfur trioxide is added in step (a) in a form stabilized with an organic sulfur compound.

23. The process according to claim 21, wherein the liquid sulfur trioxide is added in step (a) in a form stabilized with an organic sulfur compound.

24. The process according to claim 21, wherein the liquid sulfur trioxide is added in step (a) in a form stabilized with dimethyl sulfate.
